(19) United States
(12) Patent Application Publication
(10) Pub. No.: US 2016/0220834 A1
(43) Pub. Date: Aug. 4, 2016

(54) METHOD AND SYSTEM FOR SKIN TREATMENT

(71) Applicant: BTL HOLDINGS LIMITED, Limassol (CY)

(72) Inventor: TOMÁS SCHWARZ, Prague (CZ)

(21) Appl. No.: 15/060,375

(22) Filed: Mar. 3, 2016

Related U.S. Application Data
(63) Continuation-in-part of application No. 14/697,934, filed on Apr. 28, 2015.
Application No. 14/697,934 is a continuation of application No. 14/278,756, filed on May 15, 2014.
Application No. 14/278,756 is a continuation-in-part of application No. 13/297,934, filed on Nov. 16, 2011, now abandoned.
Application No. 13/297,934 is a continuation-in-part of application No. 14/038,402, filed on Sep. 26, 2013.
Application No. 14/038,402 is a continuation of application No. 13/297,608, filed on Nov. 16, 2011, now Pat. No. 8,548,599.

(51) Int. Cl. A61N 2/00; A61B 18/20; A61B 18/18; A61N 1/40; A61N 5/06

(52) U.S. Cl. CPC A61N 2/002; A61N 1/403; A61N 5/0616; A61N 5/062; A61B 18/18; A61B 18/20; A61N 2005/0661; A61N 2005/0662; A61N 2005/0659; A61N 2005/0626; A61B 2018/00464

(57) ABSTRACT

Methods are provided for remodeling and/or reducing target tissue, body contouring, tightening skin tissue and many other skin rejuvenation processes and treatments, as well as for pain relief, applying electromagnetic waves to the targeted areas of the body. The electromagnetic waves cause thermal and non-thermal effects and their parameters may be finely adjusted to achieve more indications and/or higher efficiency treatment in one session. The electromagnetic energy may be applied via one or more applicators without touching the skin.

### PRIORITY CLAIM

This application is a Continuation-in-Part of U.S. patent application Ser. No. 14/697,934, filed Apr. 28, 2015, and now pending, which a Continuation of U.S. patent application Ser. No. 14/278,756, filed May 15, 2014, and now pending, which is a Continuation-in-Part of U.S. patent application Ser. No. 13/297,934 filed Nov. 16, 2011 and now abandoned. This application is also a Continuation-in-Part of U.S. patent application Ser. No. 14/038,402 filed Sep. 26, 2013 and now pending, which is a Continuation of U.S. patent application Ser. No. 13/297,608 filed Nov. 16, 2011, now U.S. Pat. No. 8,548,599. Each of these applications is incorporated herein by reference.

### FIELD OF THE INVENTION

The present invention generally relates to a method of non-contact treatment of skin, which is also non-invasive and non-traumatic. Methods are provided for remodeling and/or reducing adipose tissue, body contouring and/or skin tightening and/or skin rejuvenation, more specifically treatment of stretch marks, cellulite, moles and/or wrinkles, and also for the treatment of vascular lesions and acne, for hair removal and/or pain relief. In particular, the invention relates to controlled treatment of the target areas of the human body using radio frequency electromagnetic waves combined with controlled application of optical electromagnetic waves. Cooling may be optionally included.

### BACKGROUND OF THE INVENTION

Human skin is composed of three basic elements: the epidermis, the dermis and the hypodermis or so called sub cutis. The dermis includes of collagen, elastic tissue and reticular fibers. The hypodermis is the lowest layer of skin and includes hair follicle roots, lymphatic vessels, collagen tissue, nerves and also subcutaneous adipose tissue. Adipose tissue is formed by aggregation of adipocytes. Most adipose tissue accumulations are caused by energy intake derived from food exceeding daily energy needs. This may result in an increase of adipocyte size and/or adipocyte number or both. Mature adipocytes may be very large, ranging up to 120 microns in diameter and containing as much as 95% lipid by volume.

Further the adipose tissue may be located in peritoneal cavity and is called visceral. In case of its excess abdominal obesity may occur. Visceral adipose tissue is located between parietal peritoneum and visceral peritoneum, closely below muscle fibers adjoining the hypodermis layer.

Excess of adipose tissue may be perceived as aesthetically undesirable. Dieting and exercise may result in reduction of adipose tissue and weight loss. However, foremost people, the adipose tissue reduction occurs rather unpredictably from all anatomical areas. This can leave the areas intended for reduction, for example, the abdomen, largely unaffected, even after significant weight loss. Various invasive and non-invasive methods have been developed to remove undesired adipose tissue from specific areas of the body.

The main invasive method is surgical-assisted liposuction, where selected volumes of adipose tissue are mechanically removed out from the patient at desired anatomical sites of the body. However, liposuction procedures are invasive and can be painful and traumatic, with many undesirable side effects and risks. Lipodissolve is another invasive procedure involving a series of drug injections intended to dissolve and permanently remove small pockets of adipose tissue from various parts of the body. It is also known as mesotherapy, lipozap, lipotherapy, or injection lipolysis. Lipodissolve also has many disadvantages and risks, to the extent that various medical associations have issued health warnings against using it.

The non-invasive methods concentrate on the acceleration of the lipolysis as the natural process of the adipose tissue reduction. This can be achieved in several ways. One of them is application of pharmaceuticals accelerating the lipolysis. However, when applied topically they tend only to affect the outermost layers of the skin, rarely penetrating to the subdermal vascular plexus. Another method uses radio frequency or ultrasound energy focused on adipose tissue to cause adipocyte destruction and its death. These methods tend to damage the melanocyte in the epidermis. The hyperthermic damage may occur in the target tissues and the body may remove the dead cellular and other debris. Non-invasive heating techniques have also been used. These techniques involve heating the adipose tissue to about 40° C. or more via direct contact with a heating element. These non-invasive methods have certain disadvantages as well, and have been used with varying degrees of success.

Apart from adipose tissue and varicose problems, the skin may be subject to many other appearance deteriorating imperfections, which may be also treated by different approach leading to more benefits described below. Additionally, other methods and apparatus have also been developed, proposing for instance irradiation of the skin by optical electromagnetic waves from near ultraviolet, visible or infrared spectra for different appearance improving effects. In recent years, combined methods using radio frequency waves and optical waves have been proposed, but these methods involve only contact radio frequency treatment.

These contact methods of radio frequency treatment have a disadvantage of creating undesirable pressure on the skin and the contact with the heating element raises accumulation of sweat on the skin surface. The salt water having a higher conductivity and/or the contact methods that use electrodes for heating the skin may easily lead to overheating and hot-spots creation which are harmful for the patient. Also, application of sole radio frequency energy does not allow varying the absorption and penetration parameters of the radiation on sufficiently large scale, thus limiting the number of indications for this therapy. Additionally, if the radio frequency waves are applied alone, the dose of heat generating electromagnetic energy must be relatively high to efficiently treat the target tissue, thus enlarging the risk of overheating, which is a serious issue for contact methods.

There is demand for improved methods and systems for target tissue treatments. The energy flow through the skin of patient needs to be improved as well to reduce or eliminate risks of overheating the skin.

### SUMMARY OF THE INVENTION

A method for treating target tissue includes positioning at least one applicator generating electromagnetic energy adjacent to the skin of a patient, but not touching the skin. Electromagnetic energy of radio frequency waves is transmitted from the at least one applicator into the target tissue. Electromagnetic energy of optical waves with frequencies in near ultraviolet, visible and infrared ranges of the spectrum is also transmitted to the skin of the patient from the at least one applicator. According to another embodiment the radio frequency waves and the optical waves are transmitted from the same applicator or applicators. The target tissue is treated by at least one type of electromagnetic waves provided. The target tissue may be remodeled. The volume and/or number of adipocytes in the adipose tissue may be reduced. The electromagnetic waves may be applied in a pulsed mode or in a continuous mode. Radio frequency and optical waves may be applied simultaneously or consecutively to the same or different skin layers or body areas.

There may be at least one source of optical waves and/or at least one source of radio frequency waves. Optical waves may be scanned across the skin. An interaction with a photosensitive molecule may be also included for some indications. This method may be also used for skin tightening and/or for remodeling collagen tissue. Another indication may include skin rejuvenation, wrinkles reduction, stretchmarks reduction, vascular lesion treatment, acne treatment, hair removal, mole removal or pain relief. The necessity of cooling the skin and biocompatibility issues is avoided by contactless application. There is also a lower risk of overheating the skin and there is no need to continuously move the applicator.

Optical waves having low or no thermal effect on the skin may be used to remodel and/or reduce adipose tissue in synergy with heating of the same target tissue by radio frequency waves. The risk of overheating is reduced. However, the method may further include cooling of the skin. The cooling may be provided in discrete intervals alternating with treating by electromagnetic waves.

Another method for treating the skin of a patient includes positioning at least one applicator adjacent to the skin of the patient, but not touching the skin. Radio frequency waves are transmitted from the at least one applicator into the target tissue continually or in discrete time intervals, treating the target tissue in discrete time intervals. The skin may be actively cooled during the time gaps when the heating is not performed.

Another method for treating the target tissue of a patient includes transmitting optical waves to the target tissue of a patient in discrete time intervals, heating the target tissue via optical waves in discrete time intervals and actively cooling the skin during the time gaps when the heating is not performed.

Combination of the optical waves effect with the radio frequency waves effect leads to a significant improvement and/or extension of indications that may be treated simultaneously to save time of both patient and health or cosmetic care provider. It provides more parameter variability to adapt the condition and/or the depth of target tissue to be treated. More target tissue and more conditions may be treated at the same time. The optical waves may enhance the desired effects of the radio frequency therapy. Use of the optical radiation may provide pain relief to the patient if used before, during or after the radio frequency treatment. Different types of energies also considerably reduce the risk of overheating the target tissue by radio frequency waves. During the treatment, the desired areas of target tissue may be maintained at predetermined temperature. To improve apoptosis and/or cell lysis (e.g. adipose cell) effect, alternative heating and cooling may also be provided.

The present methods may allow for large variability of application modes according to the condition to be treated and to the depth of the treated skin layer or layers or adipose tissue and allows treating large areas of the body with minimal need of personnel assistance during therapy.

### BRIEF DESCRIPTION OF THE DRAWINGS

FIG. 1 is a schematic diagram of a system for controlled deep treating target tissues.

FIG. 2 is a schematic view of a trans-regional course of electromagnetic field.

FIGS. 3 and 4 are schematic examples of positioning of electrodes shown in FIG. 1.

FIG. 5 is schematic diagram of an alternative electrode design.

FIG. 6 is schematic diagram of induced currents inside tissue with phase shift.

FIG. 7 is schematic diagram of induced currents inside tissue with no additional external magnetic field source.

FIG. 8 is schematic diagram of induced currents inside tissue with additional external magnetic field source.

FIG. 9 is schematic diagram of a flexible electrode arrangement in transverse cross-section.

FIG. 10 is schematic diagram of arrangement of electrodes into a matrix.

FIG. 11 is a schematic example electromagnetic energy system.

### DETAILED DESCRIPTION

Methods and systems for remodeling, reducing the volume and/or number of adipocytes, body contouring or tightening skin tissue, without contact with the skin, have now been invented. The present methods and apparatus may also be suitable for other types of appearance improving and/or pain relief treatment that can optionally be performed simultaneously or consecutively during the same session. These other types of appearance improving treatments include e.g. skin rejuvenation, wrinkles and stretch mark reduction, mole mark removal, tattoo removal, enhanced skin tightening, hair removal, treatment of vascular lesions, acne treatment, and many others.

In one of the present methods, the skin of a patient is exposed to electromagnetic waves, more precisely to a combination of radio frequency and optical waves. The optical waves include wavelengths from the near ultraviolet, visible and infrared spectrum ranges. Most preferably, the optical waves include wavelengths ranging from 405 to 1500 nm. At least one source of optical waves may be used.

According to the parameters of the electromagnetic radiation used, different layers of the skin and different tissues may be selectively treated. Various frequencies, powers and also pulse duration and repetition rates of electromagnetic radiation are applicable to provide the advantage of vast variability of penetration and absorption parameters. The practitioner can also choose the optimum treatment time for each type of electromagnetic waves and the time succession of treatments by different types of electromagnetic waves, while some of them may overlap in time. In this way, a tailor-made solution for each patient and each indication is available. The treatment can be highly selective to reduce or avoid damage of the surrounding tissues.

Combinations of more sources of electromagnetic waves allows performing the treatment of plurality of target tissue at the same time and/or treating the same target tissue simultaneously by different means, which optimizes the doses of radiation applied. This diversification also eliminates the risk of overheating, as the use of optical radiation with parameters leading to no or negligible thermic effect allows reducing the exposure to radio frequency heating without compromising the goal of the treatment. Lowering the applied radio frequency energy also leads to lower the risk of wet spots overheating, given that the low level light treatment doesn't heat the sweat. As a result, the risk of heat damage is considerably reduced.

If the patient has more imperfections to be treated situated in the same body areas, it is also possible to treat them simultaneously by different types of electromagnetic waves. Each of the electromagnetic waves may be adjusted to optimum parameters for the target tissue imperfection treatment. Thus the time of patient and of the practitioner is saved, reducing the therapy cost.

Both radio frequency waves and optical waves can be generated in pulse or in continuous mode.

The energy flux density is the output energy of the applicator divided by the treated surface area where the temperature rises to at least 37.5° C.

The sum of energy flux density of the radio frequency waves and the optical waves applied to the patient during therapy, where the therapy means simultaneous, successive or overlap treatment or treatments may last up to 120 minutes, more preferably up to 60 minutes, most preferably up to 30 minutes, is in the range of 0.0025 W·cm⁻² and 120 W·cm⁻², more preferably in the range of 0.005 W·cm⁻² and 90 W·cm⁻², most preferably in the range of 0.01 W·cm⁻² and 60 W·cm⁻². The energy flux density of optical waves constitutes at least 1%, more preferably at least 3% and most preferably at least 5% of the sum of energy flux density.

The methods described are more gentle and efficient in adipose tissue treatment or skin tightening using contactless radio frequency therapy and optical therapy. The tissue is treated by radio frequency waves and/or by optical waves. However, optical waves with no or negligible heating effects may also be advantageously used for this indication. In some indications, it may be advantageous to treat deeper adipose tissue by radio frequency waves simultaneously with the treatment of more superficial layers of the skin by optical waves leading to a photomodulation effect.

Prior art methods of radio frequency treatment generally require direct contact of an applicator with the skin. It typically also requires use of active skin cooling elements. Direct skin contact may also raise biocompatibility issues with the applicator material and further require of high sanitary standards, since the applicators are used for treatment of different patients. The practitioner must also be skilled in using the applicators since there is a risk of burning the patient.

These disadvantages are overcome by transmitting electromagnetic energy into the target tissue, without physical contact with the patient. Contactless application enables simultaneous treatments of large areas of human body. In the present contactless methods, the skin may be sufficiently cooled passively by circulating air. The application of optical waves is contactless as well.

An air gap or material with high air permeability may be placed between the skin and the applicator. This arrangement uses the human thermoregulatory system for cooling and avoids the need of artificial cooling of the skin. Optionally, the skin may be cooled via a stream of chilled or ambient temperature air. The human thermoregulatory system enables perspiration and other bodily fluids to evaporate and cool the surrounding skin. The application of electromagnetic waves is contactless, therefore sweat accumulation and/or hot spot creation are avoided. Cooling of the patient's skin may optionally use airflow circulation using a stream of cooled or ambient temperature air. Use of cooling fluids or gels is not necessary. This reduces costs and increases patient comfort. The applicator may be in direct or indirect contact with patient's skin.

Cooling can be provided by positioning an air moving device proximate to the skin. The air moving device may be attached to or implemented into the applicator. Air moving device may be any kind of fan, ventilator or blower. The blower may include an air tube connected to air source for moving air through the air tube to the patient's skin. The air source may alternatively be cooled to provide cooled air. Alternatively, air suction may be also used as an active cooling method.

There is no necessity of constant movement of the applicator of radio frequency and/or optical waves applying electromagnetic waves over a larger area. The applicator may remain in a stationary position relative to the patient for several seconds or longer, e.g. for at least 10, 30, 60, 120 or 240 seconds, or longer. The therapy treatment itself may last an hour or longer.

The temperature increase in the dermal and the sub dermal tissues also affects the triple-helix structure of collagen fibers contained in such tissues. This may result in remodeling and rejuvenation of collagen, increase of skin density and dermal thickening based on neocollagenesis. Skin tightening may also be achieved.

Remodeling and reducing the volume and/or number of adipocytes or skin tightening in the targeted areas may change the overall appearance of the body. Therefore it may be used in body contouring, body shaping and cellulite treatment.

In one aspect, the present methods selectively treat deep human tissue containing low volume of water, such as adipose tissue.

Radiant energy from the optical range of the spectrum is provided to the skin by a source of optical waves.

Radiant energy from the radio frequency range of the spectrum may be provided to the skin by at least one capacitive electrode generating an electromagnetic field. Electrode polarity may continuously fluctuate and induce an electromagnetic field inside tissue. Selective treating in the skin occurs due to dielectric losses. An inductive electrode may alternatively be used. The treatment system for creating the electromagnetic field can use bipolar electrodes, where electrodes alternate between active and return function and where the thermal gradient beneath electrodes is during treatment almost the same. The system may alternatively use monopolar electrodes, where the return electrode has sufficiently large area in contact with skin of patient and is typically positioned a relative larger distance from the active electrode. A unipolar electrode may also optionally be used.

The electromagnetic field may be applied in continuous or pulse mode. The electromagnetic field applied continuously provides a maximum amount of energy.

Using a pulse mode of radio frequency treatment, the treatment is local and the power is typically limited to about 1000 W. With the pulse mode, a high frequency field is applied in short intervals (typically in the range of 50 μs to 500 ms) and on various pulse frequencies (typically in the range of 50 to 1500 Hz). The maximum output during the continuous method is typically limited to 400 W.

Optical waves may also be applied in pulse or continuous mode. Electromagnetic energy is provided through the skin to the underlying dermal and/or subdermal tissue, without contacting the skin. The radiant energy may be converted inside the target tissue to heat. The radiant energy enables treating of the adipose tissue and/or collagen tissue, accelerating apoptosis and/or cell lysis (e.g. adipose cell), based on amount of energy transmitted to target tissue. At the same time the triple helix structure of collagen fibers may result in remodeling and/or rejuvenation of collagen, increase of skin density and dermal thickening based on neocollagenesis. In an alternative embodiment the radiant energy enables treating of target tissue resulting e.g. in neocollagenesis without adipose tissue reduction. Target tissue may be remodeled and/or reduced, therefore body contouring and/or skin tightening effect may occur.

To enhance the efficiency of the treatment in some indications, it may be advantageous to preheat the tissue by infrared radiation prior to radio frequency or combined radio frequency and optical treatment.

Optionally, an active cooling may be included. However, in many cases, auto thermoregulation by sweating is sufficient.

Simultaneous application of combined radio frequency therapy and optical therapy may reach more significant results than separate use of these therapies.

Optical radiation may be also used to attenuate the pain during or after the radio frequency treatment.

In one aspect of the invention, cells may produce heat shock proteins in response to rapid changes of thermic conditions by applied alternation of cooling and treating induced by radio frequency and/or optical waves. It has been shown that heat shock proteins stimulate reparation processes in the cells. The principles of cryolipolysis are also involved because adipocytes are more susceptible to cooling than other skin cells. By alternating the steps of cooling and treating, the apoptosis and/or cell lysis (e.g. of adipose cells) is considerably improved.

Cooling and treating, including treating by radio frequency waves, may also be used to modify and to optimally adjust the depth of penetration of particular optical radiation. If cooling is used before phototherapy, it enhances light penetration. The effects of heating in terms of light penetration are the opposite.

Optical therapy may treat the same or different skin layers as the radio frequency therapy. As mentioned above, optical therapy may also be used for multiple rejuvenation and appearance enhancing applications. Another important indication is drug-free and addiction-free pain relief in many conditions.

Non-limiting examples of optical therapies that may be preferably used in combination with the radio frequency therapy according to the present invention are: low level light therapy (LLLT), photodynamic therapy (PDT), high power laser therapy (HPLT) or intense pulsed light (IPL). However, the scope of the invention is not limited only to these particular optical irradiation methods.

Low-level light therapy is one of the methods of non-invasive rejuvenation with no or a very small thermal effect. LLLT may be effective throughout the visible, infrared and near ultraviolet spectrum ranges. The term low level refers the fact that the levels of energy or power densities are low compared to other forms of light treatment such as by lasers, which are applicable for cutting, thermal destruction or thermal coagulation. Treatment energies in LLLT are limited to 1-20 or a few J/cm2 and/or by a power of 1 mW to 500 mW per source. The depth of penetration of the low level light radiation depends on the source parameters such as wavelength, operating mode, which can be pulse or continuous, the power output, the probe design and the treatment technique. The depth of penetration where the light still has therapeutic effects should match the depth of the desired zone to be treated. The penetration depth is lower than in HPTL, up to several tens of mm approximately. Due to the low levels of absorbed energy, the treated and surrounding tissue is not heated and is not damaged. Although many wavelengths may be used, it is advantageous to use at least one beam in the visible spectrum so that the area of application on the patient's body can be easily determined by the operator.

LLLT uses either coherent light sources such as lasers or laser diodes or non-coherent light sources may include incandescent lamps, gas filled lamps, filtered lamps optimized for a particular wavelength, light-emitting diodes, etc. A combination of some of these sources types may be also used, as well as more sources of the same type.

The photons emitted by the low level sources used in LLLT therapy may be absorbed by endogenous mitochondrial chromophores in skin. Consequently, many processes are activated, as for example electron transport, increased adenosine triphosphate (ATP) production, enhanced blood micro-circulation, increase in collagen production, dermal matrix remodeling etc. LLLT can thus successfully treat a multitude of conditions that require stimulation of healing, acute/chronic pain relief or restoration of function. It has been proved that LLLT has beneficial effects on wrinkles, scars including acne scars, stimulating the scalp in hair treatment, healing of burns, skin tightening, anti-edematous effects, etc. Inflammatory skin diseases such as psoriasis and acne may be also treated by the proposed therapy. In pigmentation disorders such as vitiligo, LLLT may increase pigmentation by stimulating melanocyte proliferation.

LLLT may act also on adipose tissue reduction. It is believed that the incident light produces transient pores in adipocytes, allowing lipids to leak out into the interstitial space of adipose tissue. If the parameters are appropriate, the pores close upon cessation of the energy application and the cell membrane returns to contiguity. The adipocyte is not destroyed, but the light induced temporary opening within the cell's membrane provides a pathway for lipid to exit the cell and in the end also the patient's body. It leads to the reduction of the target tissue. This adipocyte volume reduction may restore proper adipocyte function thereby acting as an anti-diabetes mechanism.

It is advantageous to combine LLLT and radio frequency therapy for safe and efficient target tissue reduction. The simultaneous use of LLLT and radio frequency radiation allows reducing the radio frequency exposure and thus significantly reduces the risk of heat damage to the tissue.

While in LLLT the light is absorbed by endogenous cellular chromophores, PDT is based on introduction of exogenous photosensitizers into the cells which are then irradiated with wavelengths of visible or near infra-red light. Photosensitizer drugs may become activated by one or several types of optical waves. The optimal type of light depends on the target tissue and the absorption peak of the particular chromophore drug used. PDT light sources include laser, intense pulsed light, light-emitting diodes, many visible lights including natural sunlight, etc.

Unlike LLLT HPLT has pronounced thermic effects on the skin. HPLT lasers having an output of 500 mW or greater may be used for this therapy, with energy densities greater than 10 J/cm2. High power allows extremely high penetration of the light, in order of ten centimeters or even more, ensuring that the right dose actually reaches the target area localized deep in the tissue. Laser light, due to its monochromacy and coherency, can be precisely adjusted. Therefore its propagation and targeted tissue may be finely pre-defined. Research shows that tissues treated by HPLT are stimulated to increase production of a adenosine triphosphate (ATP). Similarly to LLLT, the biological responses to increased ATP production include reduction of inflammation, reduced scar tissue, increased cell metabolism, improved vascular activity, and accelerated healing. Significant improvements of many post-traumatic pathologies or osteoarthritis have been noted, as well as temporary relief of stiffness and muscle spasms. It is important to note that HPLT also provides the patients with drug-free and addiction-free acute and/or chronic mediation of pain, by decreasing inflammation and/or swelling and by increasing the release of endorphins and enkephalins. Moreover, if pulse regime is applied, the wavelength-specific photomechanical wave generated in the tissue may stimulate free nerve endings, thus blocking pain pathways in the nervous systems and bringing immediate pain relief.

High power lasers, laser diodes or intense pulse light sources (IPL) may be also used for treating pigmented targets in the skin by selective photothermolysis. The indications include e.g. vascular lesions, varicose veins, acne, pigmented lesions and mole marks or tattoos. The commonly targeted skin chromophores are hemoglobin, melanin, carbon and tattoo ink. Each chromophore has unique absorption spectrum. The wavelength of the light should match one of the absorption peaks of the targeted chromophore. The lasers or laser diodes work usually in pulse regime in these applications. The light energy absorbed by the chromophore is converted to thermal energy thereby destroying the targeted cells. Selection of the best adapted wavelength, power and pulse duration allows achieving optimal effect on targeted tissue with minimal effect on surrounding tissue.

Similar principles are used also for removal of excessive body hair. Light pulses target the hair follicle causing the hair to fall out and minimizing further growth

IPL can be used also for some other skin treatments with therapeutic or rejuvenating effects, sharing some similarities with high power laser treatment. In both cases, light is used to destroy the targets by treating. But unlike lasers that use a single wavelength of light which typically matches only one chromophore, and hence only one condition, IPL uses a broad spectrum of wavelengths. When used with filters, it may be adapted to treat various conditions. This may be achieved when the IPL operator selects the appropriate filter that matches a specific chromophore.

The figures that will be described hereinafter refer to methods of the radio frequency irradiation. Means for application of any of the optical therapy methods may be added to any of these figures. The particular setup, and especially the light source or sources used, depend on the therapeutic or aesthetic application. The basic parts of the optical irradiation system to apply the methods of the present invention include a hardware panel and a light source or multiple light sources. Non limiting examples of light sources that may be used include light emitting diodes, lasers, laser diodes, different types of lamps and filtered lamps or combinations thereof. Several light sources may be used in one apparatus. The light sources may be arranged in an array. They may be attached to each other or alternatively be individually mounted on dedicated supports. A laser scanner may be also be one of the options.

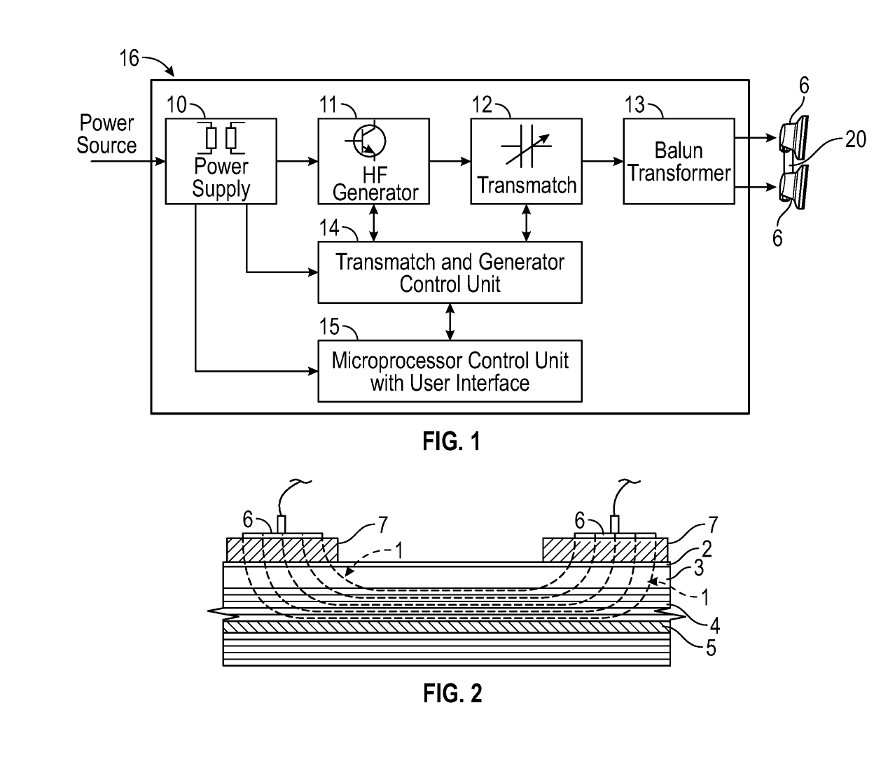

Referring now to FIG. 1, a system 16 applies electromagnetic energy through a skin layer, such as the epidermis, and to the underlying dermal and/or sub dermal tissue, and underlying collagen tissue, causing acceleration of apoptosis and/or cell lysis (e.g. adipose cell) and/or collagen remodeling. The system may include 6 blocks. The power supply 10 is connected to a power source. An HF generator (high frequency generator) 11 and a transmatch and generator control unit 14, and a microprocessor control unit with user interface 15, are connected to the power supply 10. The HF generator 11 may generate an electromagnetic field at 13.56 or 40.68 or 27.12 MHz, or 2.45 GHz or optionally at other frequencies as well. The 13.56, 27.12 and 40.68 MHz and 2.45 GHz frequencies avoid creating radio interference, as these frequencies are exclusively assigned as free or open frequencies.

The microprocessor control unit with user interface 15 provides communication between the transmatch and generator control unit 14 and user interface, which may be a touch screen on the device display.

The transmatch and generator control unit 14 receives information from the operator via the control unit and regulates the operation of the HF generator 11 and the transmatch 12. The transmatch transmits HF to a balun transformer 13, which converts unbalanced impedance to balanced impedance. This processed signal goes to two capacitive applicators 6, which may be positioned 0.5 cm or higher above the surface of the skin or applied on dielectric or insulating, non-conductive material which is in contact with the skin surface.

FIG. 2 is a schematic representation of a heat distribution under the skin. At least one applicator 6 creates an electromagnetic field. This electromagnetic field passes through the skin 2, adipose tissue 3 and muscle 4 or the bone 5. Capacitive applicators 6 provide deep heating, which heats selectively only structures with low volume of water. A spacer 7 such as a towel, gauze pad, foam pad, cloth pad and another porous or air permeable materials may be placed on the skin, with the applicator placed on the top of the spacer 7. The spacer may be made from three-dimensional material with high air permeability formed by two square fabrics with preferably low square densities connected by tough filaments. This automatically sets the separation distance between the applicator and the skin, and prevents the applicator from touching the skin. The spacer 7 may be made of various dielectric or electrically non-conductive materials. The spacer 7 may be typically dry in use. Alternatively, a reusable or a disposable spacer may be attached to the applicator. For example, the spacer may comprise posts, a frame, or other structure on the applicator that contacts the skin, while keeping the active surface of the applicator spaced apart from the skin. As described and claimed here, such spacing elements are additional elements and not part of applicator. The methods may be performed with no part or surface of the actuator in contact with the skin.

A selective heating process is observed in the dermis 3 due to dielectric losses of induced electromagnetic field. Dielectric loss is created, as part of an AC electromagnetic field power is converted to heat in the dielectric. During this process, ions accelerate and collide, polar molecules rotate, non-polar molecules undergo distortion and these movements produce thermal energy. Muscles are largely not affected by electromagnetic field 1 as they contain water and blood circulation provides cooling. Bone 5 gets little if any heating because the applicators 6 are positioned to create a field only on the upper structures. Shallow layers of the skin contain less water and are therefore heated. The adipose tissue contains the least water in comparison with the surrounding tissue and therefore the adipose tissue is heated at higher level.

Electrodes may be placed coplanar, tilted to each other or parallel to each other. Coplanar electrodes may be advantageously (but not exclusively) used for heating the shallow layers of skin. In this arrangement the electromagnetic waves tend to propagate through materials with the lowest impedance, such as epidermis and dermis. This effect may be favorably used for remodeling collagen and elastin fibers.

Electrodes tilted to each other may be advantageously used for different sized patients, limbs or another body parts. Electrodes parallel to each other with target tissue between them may be advantageously (but not exclusively) used for target tissue treatment. In this arrangement the electromagnetic waves tend to propagate more directly between electrodes. Target tissue such as adipose tissue has highest impedance and therefore transforms most of induced electromagnetic energy to heat.

In coplanar or tilted arrangement of electrodes, more distance between electrodes induces more energy in deep tissues of patient's skin, which is desirable for target tissue heating. The electrodes may be used one by one in one plane distanced at least 0.5 cm, more preferably at least 2, most preferably at least 6 cm. This may be obtained by distribution of electrodes in predetermined minimal distance or by a matrix or array of electrodes that are switched so that adjoining electrodes are not powered on at the same time. Therefore, a specified minimal distance between electrodes will be maintained. Shorter distances between electrodes may be advantageous for treatment of shallow layers of patient's skin.

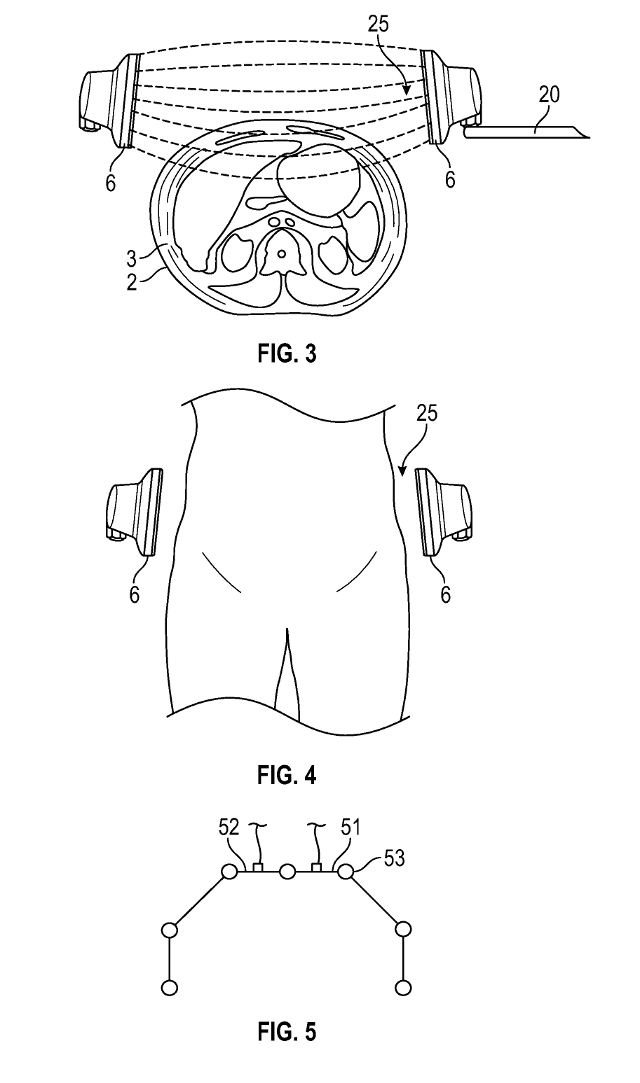

FIGS. 3 and 4 are schematic examples of positioning of the applicators or electrodes 6 providing radiant energy through the skin 2 to target tissue 3. The applicator includes at least one electrode and wiring connections to system components. The electrodes are positioned approximately 0.5 cm or higher above the surface of the skin and separated from the skin by an air gap, or placed onto a spacer 7 which is in contact with the skin surface, as shown in FIG. 2. The spacer 7, if used, may correspondingly typically be about 0.5 to 3 cm thick. The applicator 6 may be temporarily fixed in position relative to the patient, if desired, for example on a mechanical fixture or holder. It is not necessary in each instance for the applicator to be continuously moving during the procedure. This makes the procedure easier to perform, since user need not constantly keep moving the applicator over the patient's skin. Consequently, the user can accordingly simultaneously attend to other needs of a patient. The applicator 6 may have a relatively large surface area, so that the field 1 is distributed more widely through the target tissue. For example, the applicator may have a surface area of at least about 15, 30, 50, 100, or 150 cm2.

If more than one applicator is used, applicators may be positioned on opposite sides of the patient. A spacer may be positioned between at least one applicator and the skin. The electromagnetic waves may be transmitted in the range of 13.553-13.567 or 26.957-27.283 or 40.66-40.70 MHz or 2.4-2.5 GHz from the applicator into the target tissue. The temperature of treated tissue may be increased to 37-69° C., more preferably to 37-59° C., most preferably to 37-49° C.

At least one applicator may have a temperature sensor which measures and monitors the temperature of the treated target tissue. Temperature can be analyzed by a microprocessor control unit. The temperature sensor may be a contactless sensor (e.g. infrared temperature sensor), contact sensor (e.g. resistance temperature detector) or invasive sensor (e.g. a thermocouple) for exact temperature measuring of deep or shallow tissue of human skin. The microprocessor controller may use algorithms to calculate the deep or shallow temperature based on the surface temperature of the skin. A feedback system may be used to measure and control temperatures on the skin surface or below the skin surface. The feedback system may control the temperature to a predetermined level, for example by adjusting power, airflow circulation, phase shifting or supplemental magnetic field or by any combinations of thereof.

FIG. 5 illustrates arrangement of a mechanical fixture or holder for tilting electrodes which enables treatment of different sized patients, limbs or other body parts. A tilting device may include at least two electrodes 51, 52 connected by joints 53, allowing the electrodes to be spatially adjustable. Each electrode may further enable connection of additional electrodes, so that the applicator can be extended according to the needs of the particular patient. The joint connection with additional electrode(s) may be a plug and play device. The microprocessor control unit may be programmed to recognize the additional electrodes and allow the user to select a therapy with regard to the number of participating electrodes. In some embodiments the radio frequency device also enables shifting of electrodes for example by parallelogram or simply by fastening of each electrode on mechanical fixture or holder. In another embodiment the arrangement of mechanical fixture or holder enables tilting and shifting of electrodes.

Substantially coplanar electrodes may be advantageous for treatment of deep tissue. Coplanar electrodes or electrodes tilted towards each other may be used with a low impedance material placed between the electrode/s and skin of patient. The low impedance material may be laid on patient's skin. Shallow layers of patient skin may overheat during treatment with large amount of energy because the electromagnetic field tends to propagate through tissue with the lowest impedance. Supplemental low impedance material may improve the energy flow so that a relatively large amount of energy may be safely transmitted into the tissue. The material with low impedance may be a metal, alloys or other material with the same or lower impedance than epidermis and/or dermis.

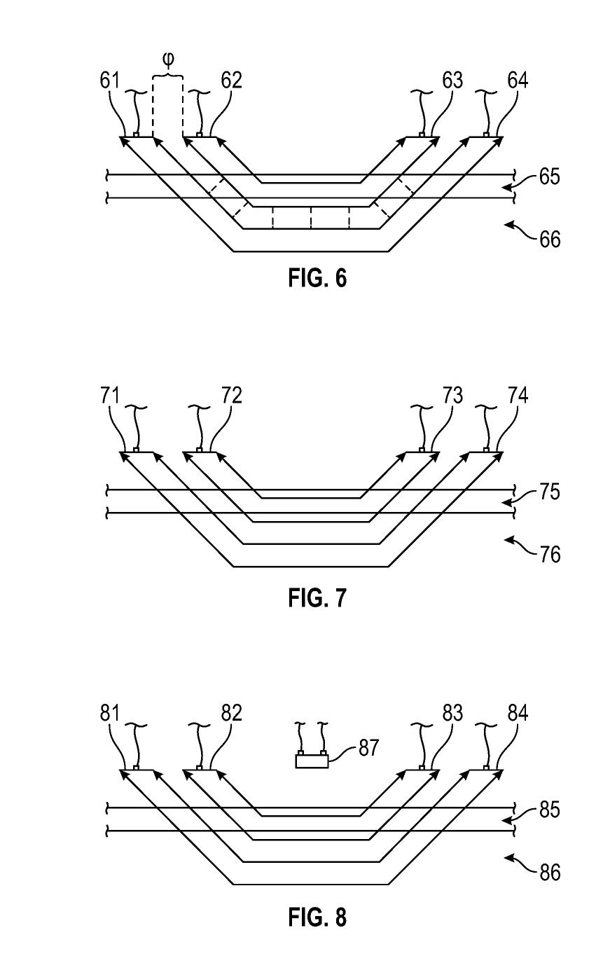

FIG. 6 shows an alternative application having phase controlled radio frequency signals which may be used to improve targeting of induced electromagnetic energy into a predetermined depth of tissue. This system may include two or more pairs of electrodes, where the first pair of electrodes 62 and 63 is inside the second pair of electrodes 61 and 64. Electrode polarity between electrodes of the inner pair fluctuates relative to the outer pair with phase shift.

In the coplanar or tilted arrangement of electrodes, a shallow layer of the skin 65 such as epidermis and dermis may be treated more when the electrodes are close together. A deep layer of the skin 66 such as hypodermis may be treated more with increasing distance between the electrodes.

As the distances between electrodes of each pair are different, each pair induces an electromagnetic field at different depths of tissue. In a coplanar or tilted arrangement of electrodes, a greater distance between the electrodes induces greater energy in deep tissues of patient's skin. With the phase shift of these pairs it is possible to control the shape of induced electromagnetic energy and therefore target tissue treatment.

In FIG. 6, the induced electromagnetic field is represented by solid line arrows, with dashed line arrows representing induced movement of charged particles caused by phase shift of induced electromagnetic fields.

Phase shift may be used in array of electrodes, where each electrode is shifted in phase separately. With phase shift it is possible to decrease the difference of potentials of adjoining electrodes and therefore decrease the amount of induced electromagnetic field in shallow layers of skin. Even if the electrodes are close together phase shift may reduce unwanted heating or overheating in shallow layers of skin.

Another system for providing targeted electromagnetic energy may use a supplemental magnetic field. FIG. 7 illustrates induced electromagnetic field inside the tissue without a supplemental magnetic field. The inner electrode pair of electrodes 72 and 73 induces an electromagnetic filed mainly inside a shallow layer of the skin 75. The outer electrode pair 71 and 74 induces an electromagnetic filed mainly in a deeper layer of the skin 76. FIG. 8 illustrates an induced electromagnetic field trajectory influenced by a permanent magnetic material or an inducing magnetic field from an electromagnet 87. The inner electrode pair of electrodes 82 and 83 induces an electromagnetic filed which is shifted from a shallow layer of the skin 85 more into a deeper layer of the skin 86. The outer electrode pair 81 and 84 induces an electromagnetic field mainly in the deeper layer of the skin 86.

An induced electromagnetic field may be deflected towards or away from the upper layers or lower layers of skin, depending on type of therapy. Based on the temperature of the skin, the microprocessor control unit may regulate the electromagnet to change the magnetic field and therefore influence the depth of the induced electromagnetic field in the skin of patient.

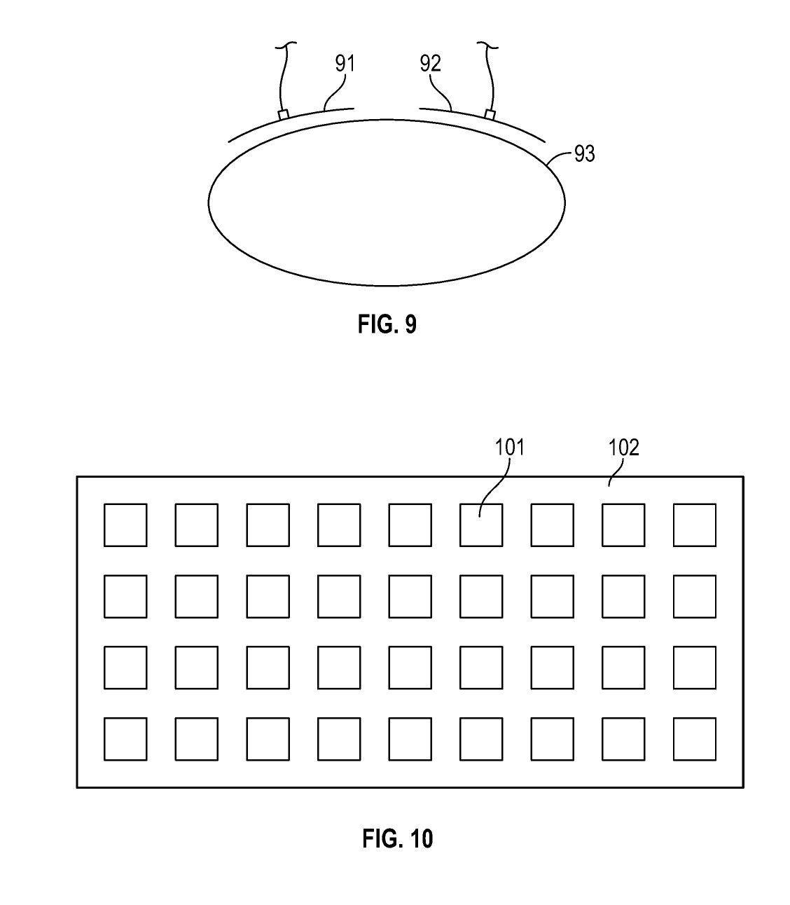

FIG. 9 illustrates a treatment system having malleable/flexible applicator having electrodes and/or light sources 91 and 92. In the embodiment, the applicator may be shaped according to the patient's shape to better match the individual. The distance between skin of the patient and applicator is therefore constant and treating of tissue is homogenous. This may help to eliminate possible temperature differences which may occur if there are any shape irregularities on human skin. Such a flexible applicator may be created from bipolar, monopolar or even unipolar system and at least one electrode. The electrode(s) may be made of flexible material to insure that the electrode(s) are the same distance from the patient's skin and substantially parallel with skin of patient.

Systems and methods may provide improved skin surface treatment for large area sections and body parts with minimal need of personnel assistance during therapy. As shown in FIG. 7, a plurality of electrodes may be are arranged adjacent to each other. The electrodes may be interconnected and partially separated from each other by a carrier surface. If the electrodes are made of rigid material the distance between these individual carrier surfaces provide high flexibility of treatment area. Alternatively, the electrodes and/or applicator may be a flexible material. The electrodes may be selectively switched on and off during treatment, optionally with the electrodes switched so that adjoining electrodes will not be powered on at the same time.

As shown in FIG. 10, a system is provided for treating large areas or parts of the body with minimal need of personnel assistance during therapy. Multiple electrodes 101 may be arranged adjacent to each other, with the electrode interconnected and partially separated from each other by carrier surface 102. If the electrodes are made of rigid material the spacing between the electrodes allows for flexible positioning of the electrodes on the body. However, preferably the electrodes are made from flexible material. The electrodes can be selectively switched on and off during treatment, optionally in a way so that adjoining electrodes are not powered on at the same time. This switching, if used, may be controlled by the microprocessor control unit and be set by the user in a user interface, or it may be set automatically based on treatment type.

Other forms of switching, such as random switching, or other algorithm switching of electrodes at specified electrode locations or distances, may also be used, to provide treatment to various depths.

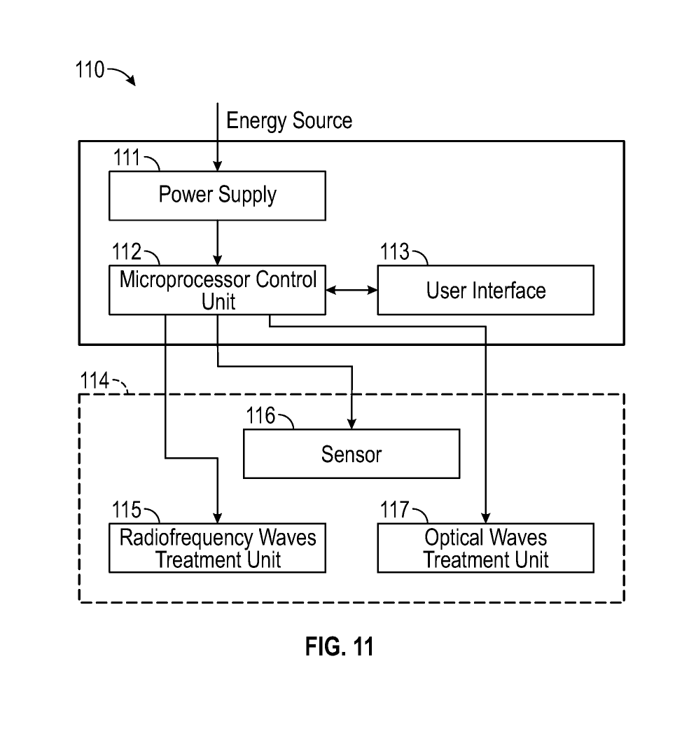

FIG. 11 shows a schematic example of a system 110 for treatment of skin by light therapy and radio frequency therapy which applies a combination of radio frequency waves and optical waves into the treated tissue. The system 110 may include a power supply 111 which is connected to an energy source. The system for skin treatment 110 includes at least one applicator 114 which may be placed inside one case or may be separated from the system for skin treatment 110 and connected by conduit. A microprocessor control unit 112 with user interface 113 may provide communication between the radio frequency waves treatment unit 115 and optical waves treatment unit 117. User interface 113 may allow setting up the treatment parameters. The radio frequency waves treatment unit 115 and optical waves treatment unit 117 may be placed in at least one applicator 114. However the treatment units may also be placed in separated applicators. The at least one applicator 114 may preferably contain a sensor unit 116. The sensor unit 116 may contain at least one sensor which senses one or more of skin temperature, skin resistance, skin color, distance of the applicator from the skin/treated tissue, electromagnetic field intensity, phase shift, current or voltage.

Any of the above mentioned sensor or sensors may measure the quantity, which may be used for controlling the therapy and based on set/pre-set limit alert the operator is informed in human perceptible form e.g. on the user interface 113. In the case of limit quantity the device may be configured to adjust output power, activate cooling or stop the therapy.

### GLOSSARY

Skin may be epidermis, dermis and hypodermis, i.e. including cutaneous and subcutaneous layers.

Target tissue may be skin and/or visceral adipose tissue.

Treatment refers to treating of target tissue by electromagnetic waves causing thermic and/or non-thermic effects.

Phototherapy may be any therapy using electromagnetic waves with wavelengths from near ultraviolet, visible and infrared spectrum range.

Optical radiation/waves may be any electromagnetic radiation/waves comprising wavelengths from near ultraviolet, visible and infrared spectrum range.

Radio frequency radiation/waves may be any electromagnetic radiation/waves having wavelengths from radio frequency spectrum range.

Electromagnetic radiation/waves may be electromagnetic radiation/waves including the entire electromagnetic spectrum.

Parameters of electromagnetic radiation/waves may be include frequency, power, pulse duration and repetition rate if applicable, and time of application.

Thus, novel methods and systems have been shown and described. Various modifications and substitutions may be made without departing from the spirit and scope of the invention. The invention, therefore, should not be limited, except by the following claims and their equivalents.

1. A method for treating the target tissue of a patient including:
positioning at least one applicator adjacent to skin of the patient, with the applicator or applicators spaced apart from the skin of the patient, and with the applicator or applicators separated from the skin by an air gap;
transmitting radio frequency waves from the applicator or from the applicators into the target tissue of the patient;
transmitting optical waves with frequency or frequencies comprised in the near ultraviolet, visible or infrared ranges of the spectrum, to the target tissue of the patient;
and treating the target tissue via at least one of the group radio frequency waves and optical waves.

2. The method of claim 1 wherein the radio frequency waves and optical waves are transmitted from the same applicator or applicators.

3. The method of claim 1 wherein the radio frequency waves and optical waves are transmitted from the at least at least one separate applicator.

4. The method of claim 1 wherein the treatment causes reduction in volume and/or number of adipose cells.

5. The method of claim 4 wherein the reduction in volume and/or number of adipose cells is caused by apoptosis and/or cell lysis.

6. The method of claim 1 wherein sum of energy flux density of the radio frequency waves and the optical waves is in the range of 0.0025 W·cm⁻² and 120 W·cm⁻².

7. The method of claim 6 wherein the optical waves constitute at least 1% of the sum of energy flux density.

8. The method of claim 6 wherein the optical waves constitute at least 3% of the sum of energy flux density.

9. The method of claim 6 wherein the optical waves constitute at least 5% of the sum of energy flux density.

10. The method of claim 1 including holding at least one applicator in a fixed position relative to the skin during at least part of the treatment.

11. The method of claim 1 including spacing the applicator 0.1-10 cm away from the skin of the patient.

12. The method of claim 1 including using the radio frequency waves either prior to, simultaneous with or after the optical waves treatment.

13. The method of claim 1 wherein the target tissue treated by radio frequency waves and optical waves do not overlap, partly overlap or entirely overlap.

14. The method of claim 1 wherein the skin to be treated by radio frequency waves is pre-heated by optical waves.

15. The method of claim 1 wherein at least one applicator has a surface area of at least 15 cm².

16. The method of claim 1 wherein the optical waves are scanned across the skin.

17. The method of claim 1 further including administration of a photosensitive molecule prior to transmitting optical waves.

18. The method of claim 1 wherein the temperature of target tissue is increased to 37-69° C.

19. The method of claim 1 wherein the optical waves have low or no thermal effect on the skin.

20. The method of claim 1 further including cooling of the skin.

21. The method of claim 20 wherein the cooling is repeatedly stopped and resumed.

22. The method of claim 1 including measuring one or more of skin temperature, skin resistance, skin color, distance of the applicator from the skin/treated tissue, electromagnetic field intensity, phase shift, current and/or voltage.

23. The method of claim 22 including informing the operator in human perceptible form when measure values are out of a set/pre-set limit.

24. The method of claim 23 including adjusting output power, activating cooling and/or stopping the therapy when measure values are out of the set/pre-set limit.

25. The method of claim 1 further comprising:
transmitting optical waves and/or or radio frequency waves to the skin of the patient in discrete time intervals; heating the skin via optical waves and/or radio frequency waves in discrete time intervals; and actively cooling the skin during the time gaps when the heating is not performed.

26. The method of claim 1 with the applicator including a flexible applicator.

27. The method of claim 1 further including first and second applicators and switching the first and second applicators on and off.

28. The method of claim 1 where the radio frequency waves transmitted by the applicators are phase shifted.

29. The method of claim 1 including exposing tissue to a supplemental magnetic field.
